(19) United States
(12) Patent Application Publication
(10) Pub. No.: US 2024/0415805 A1
(43) Pub. Date: Dec. 19, 2024

(54) COMPOSITIONS AND METHODS OF TREATING INVASIVE PATHOGENS

(71) Applicants: Meharry Medical College, Nashville, TN (US); Duke University, Durham, NC (US)

(72) Inventors: Hua XIE, Nashville, TN (US); Chin-Ho CHEN, Durham, NC (US); Li HUANG, Durham, NC (US)

(73) Assignees: Meharry Medical College, Nashville, TN (US); Duke University, Durham, NC (US)

(21) Appl. No.: 18/744,161

(22) Filed: Jun. 14, 2024

Related U.S. Application Data
(60) Provisional application No. 63/472,968, filed on Jun. 14, 2023.

(51) Int. Cl. A61K 31/357; A61K 31/4164; A61K 45/06; A61P 1/02; A61P 31/04

(52) U.S. Cl. CPC A61K 31/357; A61K 31/4164; A61K 45/06; A61P 1/02; A61P 31/04

(57) ABSTRACT

The disclosure provides compounds that inhibit the invasion of host cells by intracellular pathogens. The disclosure also relates to use of such compounds in methods of treating and preventing periodontitis or a periodontitis-related condition or symptom.

### CROSS-REFERENCE TO RELATED APPLICATIONS

This application claims the benefit of and priority to U.S. Provisional Application No. 63/472,968, filed on Jun. 14, 2023, and entitled “Compositions and Methods of Treating Invasive Pathogens,” the disclosure of which is expressly incorporated by reference in its entirety.

### FIELD OF THE DISCLOSURE

The present disclosure relates generally to methods of treatment and/or prevention of diseases and medical conditions, and specifically to those diseases and medical conditions that are associated with or characterized by invasive pathogens, such as those involved with certain periodontal diseases.

### BACKGROUND

The microbiome plays a vital role in human health, is involved in digestion and the immune responses, and is associated with infectious and dysbiotic diseases. Modulation of the microbiota including elimination of keystone pathogens represents a potential therapeutic strategy for the prevention or treatment of microbiome-associated diseases. Porphyromonas gingivalis (Pg) is a gram-negative bacterium that represents a keystone pathogen of periodontitis, which is the 6^{th} most common infection worldwide, with an estimated 5-20% of the population suffering from chronic periodontitis. Pg contributes to periodontitis by transforming commensal microbial communities into pathogenic communities that induce a breakdown of periodontal tissue-microbe homeostasis and leads to uncontrolled inflammation and tissue destruction.

Bacterial invasion is a process in which bacteria, such as Pg, actively enter non-phagocytic cells, such as epithelial cells. In general, invasive bacteria express surface invasins that interact with corresponding receptors on the surface of non-phagocytic cells. As a result, a cascade of signaling events in the host cells is triggered, including protein phosphorylation, cytoskeleton rearrangements, membrane extension, and bacterial engulfment. After internalization, bacteria can survive and multiply in the cytosol as well as spread to other host cells. The ability to invade host cells empowers bacteria to establish a successful infection by avoiding immune responses and antibiotic treatment. Intracellular bacteria also are known to contribute to persistence of chronic infections, such as by serving as opportunity bacteria.

For instance, Pg possesses a vast array of effector molecules and systems that enable its survival in the oral cavity. One of Pg's features is its ability to invade and survive within host cells such as fibroblasts, epithelial, and endothelial cells. Certain membrane proteins involved in Pg attachment to, and invasion of, host cells include FimA, hemagglutinin A, and heat-stress protein. Pg strains with invasion deficiency can exhibit approximately 10 times less activity. Invasive ability is important in establishing Pg infection in the oral cavity. However, studies on bacterial invasion inhibitors are limited.

Conventional therapies for periodontitis, including scaling and root planing cannot prevent the re-emergence of a pathogenic microbial community, as high levels of Pg have been observed at the periodontal sites of patients with refractory periodontitis. Systemic or local administration of antibiotics may be used adjunctively to combat severe and refractory manifestations of the disease, but recommended dosing for the treatment does not eliminate intracellular Pg which may proliferate intracellularly and spread to adjacent cells. Indeed, difficulties in eliminating Pg from the oral cavity are due, at least in part, to Pg's ability to invade host cells and the drug-resistant nature of dental plaques, which facilitate the organism in evading host defense systems and antibiotics. In addition, there are increasing public health concerns regarding the use of antibiotics, including disruption of the symbiotic or mutualistic relationships between host and commensal microbiota and emerging bacterial resistance to antibiotics. Further, current treatment strategies are often effective only temporarily, and recurrence of the disease is common,

Therefore, there is a need in the art for therapeutic agents that can specifically eliminate intracellular Pg from the oral cavity.

### SUMMARY

In certain embodiments, this disclosure provides compounds that inhibit the invasion of host cells by one or more intracellular pathogens. These find use, for example, in treating periodontitis or a periodontitis-related condition or symptom, preventing periodontitis or a periodontitis-related condition or symptom, reducing the likelihood of periodontitis or a periodontitis-related condition or symptom, reducing the severity of periodontitis or a periodontitis-related condition or symptom, or a combination thereof.

In a first aspect, a pharmaceutical composition for inhibiting invasion of a pathogen into a host cell is disclosed. In embodiments, the pharmaceutical composition includes a therapeutically effective amount of a protein kinase C beta (PKC-β) agonist and a pharmaceutically acceptable carrier, wherein the pharmaceutical composition is formulated for local delivery to a gingival tissue in a subject. The PKC agonist can include a PKC-β agonist. In one embodiment, the PKC-β agonist includes gnidimacrin or a pharmaceutically acceptable derivative thereof. In some embodiments, the therapeutically effective amount of the PKC-β agonist is sufficient to achieve a sub nanomolar concentration in the gingival tissue of the subject. The therapeutically effective amount of the PKC-β agonist can be sufficient to achieve a concentration of at least about 10 nM of gnidimacrin in the gingival tissue of the subject. In embodiments, the therapeutically effective amount of the PKC-β agonist is sufficient to achieve a concentration of up to about 1000 nM of gnidimacrin in the gingival tissue of the subject. The therapeutically effective amount of the PKC-β agonist can be sufficient to achieve a concentration of up to about 100 nM gnidimacrin in the gingival tissue of the subject. In one embodiment, the therapeutically effective amount of the PKC-β agonist is sufficient to achieve a concentration of about 20 nM gnidimacrin in the gingival tissue of the subject.

In certain embodiments, the subject has been diagnosed with periodontitis, a periodontitis-related condition, or a periodontitis-related symptom. In one embodiment, the pathogen includes a bacterium. The bacterium can be Porphyromonas gingivalis.

In some embodiments, the pharmaceutical composition, includes a therapeutically effective amount of an antibiotic. The antibiotic can include gentamicin, metronidazole, or a combination thereof.

In one embodiment, the pharmaceutical composition includes a dentifrice. The dentifrice can include a paste, a gel, a mouthwash, a powder, a tooth soap, or a combination thereof.

In a second aspect, a method of treating or preventing bacterial invasion in a non-phagocytic cell in a subject is disclosed. In embodiments, the method includes administering to the subject a therapeutically effective amount of a compound including a PKC-β agonist. The non-phagocytic cell can include an epithelial cell, an endothelial cell, a fibroblast, a smooth muscle cell, or a combination thereof. The epithelial cell can include an oral epithelial cell. In one embodiment, the oral epithelial cell includes a human oral keratinocyte (HOK). The endothelial cell can include an aortic endothelial cell, a heart endothelial cell, or a combination thereof. In embodiments, the fibroblast includes a gingival fibroblast.

In certain embodiments, the subject suffers from an infection of Porphyromonas gingivalis. The subject can be at risk from an infection of Porphyromonas gingivalis. In one embodiment, the PKC agonist includes a PKC-β agonist. The PKC-β agonist can include gnidimacrin or a pharmaceutically acceptable derivative thereof.

In embodiments, the therapeutically effective amount of the PKC-β agonist is sufficient to achieve a concentration of at least about 10 nM of gnidimacrin in the gingival tissue of the subject. The therapeutically effective amount of the PKC-β agonist can be sufficient to achieve a concentration of up to about 1000 nM of gnidimacrin in the gingival tissue of the subject. In one embodiment, the therapeutically effective amount of the PKC-β agonist is sufficient to achieve a concentration of about 20 nM gnidimacrin in the gingival tissue of the subject.

In certain embodiments, the method further includes administering a therapeutically effective amount of an antibiotic. The antibiotic can include gentamicin, metronidazole, or a combination thereof.

A third aspect of the present disclosure includes a method of treating or preventing periodontitis, a periodontitis-related condition, or a periodontitis-related symptom in a gingival tissue of a subject in need thereof, including locally administering a compound including a therapeutically effective amount of a PKC-β agonist to the gingival tissue of the subject. The periodontitis, periodontitis-related condition, or periodontitis-related symptom can be bacterially mediated. In one specific embodiment, the periodontitis, periodontitis-related condition, or periodontitis-related symptom is mediated by Porphyromonas gingivalis.

In certain embodiments of the method, the compound is administered in a dentifrice. The dentifrice includes a paste, a gel, a mouthwash, a powder, a tooth soap, or a combination thereof. The compound can include an activator of PKC-β. In one embodiment, the compound includes gnidimacrin or a pharmaceutically acceptable derivative thereof.

In certain embodiments, the gnidimacrin or pharmaceutically acceptable derivative thereof is administered in a composition containing at least 20 nM gnidimacrin or a pharmaceutically acceptable derivative thereof. The therapeutically effective amount of the compound can be administered with a pharmaceutically acceptable carrier. In one particular embodiment, the compound includes gnidimacrin, and the therapeutically effective amount is sufficient to achieve a concentration of up to about 1000 nM gnidimacrin in tissue of the subject. The therapeutically effective amount can be sufficient to achieve a concentration of up to about 100 nM gnidimacrin in tissue of the subject. In an exemplary embodiment, the therapeutically effective amount is sufficient to achieve a concentration of about 20 nM gnidimacrin in a gingival tissue of the subject. The method can further include administering a therapeutically effective amount of an antibiotic.

In a fourth aspect, the disclosure relates to a method of reducing the likelihood of invasion of a host cell by an intracellular bacterium including contacting the host cell with an effective concentration of a PK-β agonist. The method can further include contacting the host cell with an effective concentration of an antibiotic. In one embodiment, the PKC-β agonist includes gnidimacrin or a pharmaceutical derivative thereof.

In one embodiment, the therapeutically effective amount of the PKC-β agonist is about 20 nM gnidimacrin or the pharmaceutical derivative thereof. The bacterium can be Porphyromonas gingivalis. In certain embodiments, the host cell includes an epithelial cell, an endothelial cell, a fibroblast, a smooth muscle cell, or a combination thereof. The epithelial cell can include an oral epithelial cell. In one embodiment, the oral epithelial cell includes a human oral keratinocyte (HOK). The endothelial cell can include an aortic endothelial cell, a heart endothelial cell, or a combination thereof. In one embodiment, the fibroblast includes a gingival fibroblast.

A fifth aspect of the present disclosure includes a pharmaceutical composition for treating or preventing periodontitis or a periodontitis-related condition or symptom in a subject, the pharmaceutical including a therapeutically effective amount gnidimacrin or a pharmaceutical derivative thereof, a therapeutically effective amount of an antibiotic compound, and a pharmaceutically acceptable carrier, wherein the pharmaceutical composition is formulated for local delivery to gingival tissue in the subject. In some embodiments, the therapeutically effective amount of the gnidimacrin or the pharmaceutical derivative thereof is sufficient to achieve a concentration of about 10 nM to about 100 nM gnidimacrin or pharmaceutical derivative thereof in the gingival tissue of the subject. In another embodiment, the antibiotic includes gentamicin, metronidazole, or a combination thereof. In still another embodiment, the gnidimacrin or the pharmaceutical derivative thereof is formulated in a dentifrice and the dentifrice includes a paste, a gel, a mouthwash, a powder, a tooth soap, or a combination thereof.

The above presents a simplified summary to provide a basic understanding of some aspects of the claimed subject matter. This summary is not an extensive overview. It is not intended to identify key or critical elements or to delineate the scope of the claimed subject matter. Its sole purpose is to present some concepts in a simplified form as a prelude to the more detailed description that is presented herein.

### BRIEF DESCRIPTION OF THE DRAWINGS

Further features and advantages can be ascertained from the following detailed description that is provided in connection with the drawings described below:

FIG. 1 shows the chemical structure of gnidimacrin (GM) according to one embodiment of the present disclosure.

FIGS. 2A-2B provide bar graphs showing inhibition of Pg invasion and elimination of intracellular Pg by GM. In FIG. 2A, each bar represents relative human oral keratinocyte (HOK) invasion rate of Pg in the presence of different concentrations of GM. In FIG. 2B, each bar represents the relative level of intracellular Pg in HOKs in the presence of GM or antibiotics. An asterisk indicates the statistical significance between invasive rates or intracellular levels in the presence or absence of GM (P<0.05; t test).

FIGS. 3A-3E provide confocal microscopy images showing that GM blocks invasion of Pg 33277 vesicles into human oral keratinocytes (HOKs) in a dose-dependent matter. HOKs were seeded in the glass-bottomed dishes and treated with PBS (control, FIG. 3A), Pg vesicles for 30 min (FIG. 3B), Pg vesicles in the presence of GM at 5 nM (FIG. 3C), 10 nM (FIG. 3D), or 20 nM (FIG. 3E). The HOKs were stained with polyclonal antibodies, then anti-IgG with Alex Fluor 546 (red) and DAPI (blue) and visualized under a confocal microscope. Scale bar=20 μm for the panel.

FIG. 4 shows a graphical comparison of Pg growth in the presence or absence of GM. Pg 33277 were grown in TSB in the presence or absence of GM. OD600 was measured after growth for 16 h. Bars are means of triplicate samples, with error bars representing the standard deviation.

FIG. 5 shows Western blot images used for characterization of the effects of GM on PKC activation in activated human primary CD4 cells under one embodiment. Anti-CD3/CD28 (1 ug/ml) activated human primary CD4 cells were cultured in the presence or absence of GM (1 nM) for 12 hours. The cytosolic fraction of the cells was analyzed with 10% SDS PAGE followed by Western blot analysis using a panel of monoclonal antibodies against PKC isozymes (Santa Cruz Biotech).

FIG. 6 shows a graphical representation of luciferase levels as an indicator of cell viability in HOKs treated with GM. Each bar represents the mean±standard deviation of luminescence levels representing relative the number of viable cells. Data are the mean of at least three independent experiments.

FIGS. 7A-7B show confocal microscopic images of microtubule rearrangement in HOKs induced by GM. HOKs were treated with GM (20 nM) for 0 minutes (FIG. 7A), or 30 minutes (FIG. 7B) and were stained with anti-α-tubulin, anti-IgG with Alex Fluor 546 (red) and DAPI (blue) and visualized under a confocal microscope. Scale bar, 20 μm.

### DETAILED DESCRIPTION

### A. Definitions

Unless otherwise defined, all terms (including technical and scientific terms) used herein have the same meaning as commonly understood by one of ordinary skill in the art of this disclosure. It will be further understood that terms, such as those defined in commonly used dictionaries, should be interpreted as having a meaning that is consistent with their meaning in the context of the specification and should not be interpreted in an idealized or overly formal sense unless expressly so defined herein. Well known functions or constructions may not be described in detail for brevity or clarity.

The terminology used herein is for the purpose of describing particular embodiments only and is not intended to be limiting. As used herein, the singular forms “a”, “an” and “the” are intended to include the plural forms as well, unless the context clearly indicates otherwise.

The terms “first,” “second,” and the like are used herein to describe various features or elements, but these features or elements should not be limited by these terms. These terms are only used to distinguish one feature or element from another feature or element. Thus, a first feature or element discussed herein could be termed a second feature or element, and similarly, a second feature or element discussed herein could be termed a first feature or element without departing from the teachings of the present disclosure.

The term “consisting essentially of” means that, in addition to the recited elements, what is claimed may also contain other elements (steps, structures, ingredients, components, etc.) that do not adversely affect the operability of what is claimed for its intended purpose as stated in this disclosure. Importantly, this term excludes such other elements that adversely affect the operability of what is claimed for its intended purpose as stated in this disclosure, even if such other elements might enhance the operability of what is claimed for some other purpose.

The terms “about” and “approximately” shall generally mean an acceptable degree of error or variation for the quantity measured given the nature or precision of the measurements. Typical, exemplary degrees of error or variation are within 20 percent (%), preferably within 10%, and more preferably within 5% of a given value or range of values. For biological systems, the term “about” refers to an acceptable standard deviation of error, preferably not more than 2-fold of a given value. Numerical quantities given herein are approximate unless stated otherwise, meaning that the term “about” or “approximately” can be inferred when not expressly stated.

Terms such as “administering” or “administration” include acts such as prescribing, dispensing, giving, or taking a substance such that what is prescribed, dispensed, given, or taken actually contacts the patient's body externally or internally (or both). In embodiments of this disclosure, terms such as “administering” or “administration” include self-administering, self-administration, and the like, of a substance. Indeed, it is specifically contemplated that instructions or a prescription by a medical professional to a subject or patient to take or otherwise self-administer a substance is an act of administration.

The terms “prevention,” “prevent,” and “preventing,” as used herein, refer to a course of action (such as administering a pharmaceutical composition) initiated prior to the onset of a clinical manifestation of a disease state or condition so as to reduce the likelihood or severity. Such reduction in likelihood or severity need not be absolute to be useful.

The terms “treatment,” “treat,” and “treating” as used herein refer to a course of action (such as administering a pharmaceutical composition) initiated after the onset of a clinical manifestation of a disease state or condition so as to eliminate or reduce such clinical manifestation of the disease state or condition. Such treating need not be absolute to be useful.

The term “in need of treatment” as used herein refers to a judgment made by a caregiver that a patient requires or will benefit from treatment. This judgment is made based on a variety of factors that are in the realm of a caregiver's expertise, but that include the knowledge that the patient is ill, or will be ill, as the result of a condition that is treatable by a method or composition of the present disclosure.

The term “in need of prevention” as used herein refers to a judgment made by a caregiver that a patient requires or will benefit from prevention. This judgment is made based on a variety of factors that are in the realm of a caregiver's expertise, but that include the knowledge that the patient will be ill or may become ill, as the result of a condition that is preventable by a method or composition of the disclosure.

The term “individual”, “subject” or “patient” as used herein refers to any animal, including mammals, such as mice, rats, other rodents, rabbits, dogs, cats, swine, cattle, sheep, horses, primates, and humans. The term may specify male or female or both, or exclude male or female.

Terms such as “comprise” and “include” as used herein are inclusive, and non-exclusive, and should therefore be construed to mean “comprise/include but are not limited to.” Permissive and optional terms such as “may,” “can,” “some embodiments,” and the like, as used herein, are also inclusive and non-exclusive.

The terms “inhibit,” “decrease,” and/or “reduce the likelihood of” (and like terms) generally refer to the act of reducing, either directly or indirectly, a function, activity, or behavior relative to the natural, expected, or average or relative to current conditions. It is understood that this is typically in relation to some standard or expected value, in other words, it is relative, but that it is not always necessary for the standard or relative value to be referred to. Such terms can include complete inhibition, complete reduction, or elimination of the likelihood of a function, activity, or behavior relative to the natural, expected, or average or relative to current conditions.

The term “pharmaceutically acceptable carrier” refers to one or more compatible solid or liquid fillers, diluents, or encapsulating substances that do not cause significant irritation to a human or other vertebrate animal and do not abrogate the biological activity and properties of the administered compound. Such carriers include, but are not limited to, vehicles, adjuvants, surfactants, suspending agents, emulsifying agents, inert fillers, diluents, excipients, wetting agents, binders, lubricants, buffering agents, disintegrating agents and carriers, as well as accessory agents, such as, but not limited to, coloring agents and flavoring agents (collectively referred to herein as a carrier). Typically, the pharmaceutically acceptable carrier is chemically inert to the active compounds and has no detrimental side effects or toxicity under the conditions of use. The pharmaceutically acceptable carriers can include polymers and polymer matrices. The nature of the pharmaceutically acceptable carrier may differ depending on the particular dosage form employed and other characteristics of the composition.

The term “therapeutically effective amount” as used herein refers to an amount of a compound, either alone or as a part of a pharmaceutical composition, that is capable of having any detectable effect to alleviate any symptom, aspect, or characteristics of a disease state or condition. Such effect need not be absolute to be beneficial.

The term “prodrug” as used herein includes functional derivatives of a disclosed compound which are readily convertible in vivo into the required compound. Thus, in the methods of treatment of the present disclosure, the term “administering” shall encompass the treatment of the various disease states/conditions described with the compound specifically disclosed or with a prodrug which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

The term “pharmaceutically acceptable salts” as used herein includes salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, oxalic, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The term “pathogen,” as used herein, can mean any agent that is capable of entering a host cell and causing a condition or disease in a subject. In embodiments, the word “pathogen” refers to an organism. A pathogen can include a bacterium, fungus, virus, viroid, protist, prion, protozoan, or a combination thereof. Non-limiting examples of bacteria include P. gingivalis, Bartonella henselae, Francisella tularensis, Listeria monocytogenes, Salmonella typhi, Brucella spp., Legionella spp., Mycobacterium spp., Nocardia spp., Rhodococcus equi, Yersinia spp., Neisseria meningitidis, Chlamydia spp., Rickettsia spp., and Coxiella spp. In one embodiment, the intracellular bacteria is P. gingivalis. The pathogen can be any oral pathogen, regardless of its taxon. In one embodiment, the pathogen is an intracellular, parasitic fungus. Examples of intracellular parasitic fungi include, but are not limited to, Histoplasma capsulatum, Cryptococcus neoformans, and Pneumocystis jirovecii. Protozoal examples include apicomplexans (e.g., Babesia spp., Plasmodium spp., Cryptosporidium parvum, Cyclospora cayetanensis, Isospora belli, and Toxoplasma gondii) and trypanosomatids.

“Host cell,” as used herein can include any cell that is vulnerable to invasion by a pathogen. The host cell can be disposed within, obtained from, or otherwise derived from a subject. Exemplary host cells include those from various mammalian species, including humans and domesticated animals. In embodiments, the host cell includes a non-phagocytic cell. In embodiments, the host includes a non-phagocytic cell. Non-limiting examples of a host cell include such as fibroblasts, epithelial, and endothelial cells. In embodiments, the host cell is an oral epithelial cell, a gingival fibroblast, an aortic endothelial cell, a heart endothelial cell, or a vascular smooth muscle cell. Such cells are vulnerable to various intracellular bacteria, including the aforementioned P. gingivalis. In more specific embodiments, the host cell is a cell from a subject's gingival tissue selected from the group consisting of an oral epithelial cell and a gingival fibroblast. In further specific embodiments, the host cell is an oral keratinocyte, such as an oral keratinocyte from a subject.

### B. Compounds

The present disclosure provides a compound that inhibits the invasion of host cells by intracellular pathogens, which can be referred to herein as an “active compound.” In embodiments, the disclosure includes one or more active compounds that inhibit the invasion of bacteria into one or more host cells of a subject. In certain embodiments, the active compound can prevent formation of a reservoir of one or more intracellular pathogens in a host cell.

In embodiments, the active compound includes an inhibitor of any of the events that lead to bacterial engulfment into a host cell. In one embodiment, the active compound inhibits the binding of one or more invasins (which can be expressed on the surface of invasive bacteria) to associated receptors on one or more non-phagocytic cells.

The active compound can inhibit any one or more of the following events that lead to intracellular engulfment of a pathogen: protein phosphorylation, cytoskeleton rearrangement, membrane extension, vesical formation, and endocytosis. In embodiments, the active compound inhibits any one or more of the exemplary events either in vitro or in vivo. The present disclosure provides compounds that directly decrease any one or more of such events, indirectly decrease any one or more of such events, or a combination thereof. In embodiments, indirect modulation of one or more events is accomplished by stimulating the activity of a molecule that decreases the event, inhibiting the activity of a molecule that increases the event, or a combination thereof. In embodiments, the active compound interferes with receptor-mediated endocytosis of an invasive pathogen. In various embodiments, the active compound includes an activator of protein kinase C (PKC). In one embodiment, the active compound includes a PKC-β agonist. In embodiments, the active compound activates PKC-β in a dose-dependent manner.

The active compounds can have downstream effects of treating and preventing periodontitis or a periodontitis-related condition or symptom in a subject. As discussed above, Porphyromonas gingivalis (Pg) represents a keystone pathogen of periodontitis that multiplies in host cells and in the extracellular matrix, creating reservoirs for a persistent infection. Therefore, without being bound by any theory, active compounds that target Pg invasion of host cells can prevent or reverse the progress of periodontitis.

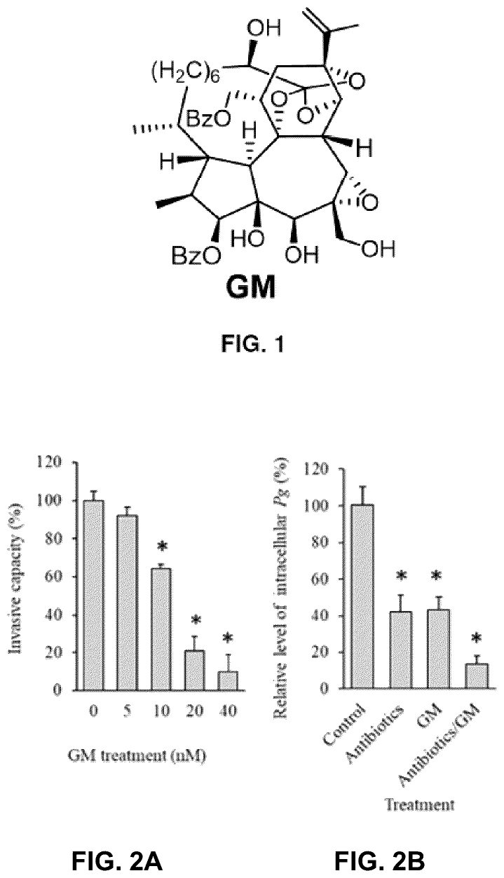

In embodiments, the active compound includes a diterpene. One specific example of an active compound includes gnidimacrin (GM). GM is a macrocylic diterpenoid ester that can be isolated from Stellera chamaejasme. In embodiments, the active compound has the chemical structure shown in FIG. 1. As explained in more detail in the working examples herein, the active compound can effectively block Pg and its vesicles from invasion into human cells. In embodiments, inhibition of Pg with an active compound is observed at a low, nanomole concentrations of GM. In certain embodiments, Pg inhibition occurs with active compound concentrations of at least about 10 nM. For example, in some embodiments, Pg inhibition occurs with active compound concentrations of at least about 20 nM. Further, the present disclosure reveals that GM does not affect host cell viability. In combination, the presently disclosed findings suggest that potent inhibitory activity and low cytotoxicity make GM an ideal candidate for anti-Pg drug development.

Unless stated otherwise, all active compounds are to be construed as including a metabolite; such a metabolite may be formed either in vivo within the body or as a result of biochemical activity in vitro. It is within the scope of this disclosure than any active compound may be limited to a non-metabolite compound.

### C. Pharmaceutical Compositions

One embodiment includes a pharmaceutical composition that includes the active compound. The compositions disclosed may include one or more of such active compounds, in combination with a pharmaceutically acceptable carrier. For instance, in one embodiment, the present disclosure provides a pharmaceutical composition including a PKC-β agonist and a pharmaceutically acceptable carrier. In another embodiment, the present disclosure provides a pharmaceutical composition including gnidimacrin and a pharmaceutically acceptable carrier. Examples of pharmaceutically acceptable carriers and methods of formulation may be found in Remington: The Science and Practice of Pharmacy (20^{th} Ed., Lippincott, Williams & Wilkins, Daniel Limmer, editor), and are generally well understood by those skilled in the art. To form a pharmaceutically acceptable composition suitable for administration, such compositions can contain a therapeutically effective amount of an active compound.

The pharmaceutical compositions of the disclosure can be used in the treatment and prevention methods of the present disclosure. Such compositions are administered to a subject in amounts sufficient to deliver a therapeutically effective amount of the active compound so as to be effective in the treatment and prevention methods disclosed herein. The therapeutically effective amount may vary according to a variety of factors such as the subject's condition, weight, sex and age. For example, some embodiments of the composition include up to the median lethal dose (LD₅₀) of the active compound. The LD₅₀ can be ascertained using standard toxicological methods, or by reference to past studies.

The therapeutically effective amount of the pharmaceutical composition may be formulated to achieve a desired concentration of the active compound in the gingival tissue of the subject. In some embodiments, the composition contains a therapeutically effective amount of the active compound that is sufficient to achieve a concentration of at least about 1 nM active compound in the gingival tissue of a subject. In some embodiments, the composition contains an amount of active compound sufficient to achieve a concentration of up to about 10 μM, up to about 20 μM, up to about 30 μM, or up to about 40 μM in the gingival tissue of a subject. In one embodiment, the therapeutically effective amount of the active compound includes an amount sufficient to achieve a concentration of at least about 20 nM active compound in the gingival tissue of a subject. The composition can contain an amount of active compound sufficient to achieve a concentration of between about 5 nM and 1 μM active compound in the gingival tissue of a subject. In specific embodiments, the composition contains an amount of active compound sufficient to achieve a concentration of at least about 10 nM, about 20 nM, about 30 nM, about 40 nM, about 50 nM, about 60 nM, about 70 nM, about 80 nM, about 90 nM, and about 100 nM in the gingival tissue of a subject. In one specific embodiment the pharmaceutical composition contains an amount of GM sufficient to achieve a concentration of at least about 5 nM, at least about 10 nM, at least about 15 nM, at least about 20 nM, at least about 25 nM, at least about 30 nM, at least about 35 nM, or at least about 40 nM in a subject's gingival tissue. In certain embodiments, the composition contains an amount of active compound sufficient to achieve a concentration of up to 1 nM, up to 5 nM, up to 10 nM, up to 15 nM, up to 20 nM, up to 25 nM, up to 30 nM, up to 40 nM, up to 50 nM, up to 60 nM, up to 70 nM, up to 80 nM, up to 90 nM, and up to 100 nM in the gingival tissue of a subject. In some embodiments, the composition contains an amount of active compound sufficient to achieve a sub nanomolar concentrations. The concentration of active compound in the composition can be less than about 1 nM.

In embodiments, the active compound can be co-administered with an antibiotic or a combination of two or more antibiotics. At least one antibiotic and the active compound can be provided in a single pharmaceutical composition or as separate pharmaceutical compositions or formulations. One embodiment includes a pharmaceutical composition that includes a therapeutically effective amount of an active compound and a therapeutically effective amount of an antibiotic. In certain embodiments, co-administration of an antibiotic with a pharmaceutical composition including an active compound generally increases the therapeutic effectiveness of the active compound. In other words, including an antibiotic with a pharmaceutical composition that includes an active compound can produce a synergistic effect that allows for greater inhibition of pathogenic invasion into a host cell. In some embodiments, bidirectional synergy is generally seen. That is, the combination of the two active agents—the antibiotic and the active compound—gives rise to inhibitory activity that is higher than that of either agent alone.

In some respects, the amount of the active compound in the pharmaceutical composition can be altered based on the amount of the antibiotic given to the subject. For example, in certain embodiments where the pharmaceutical composition includes an antibiotic, a lower concentration of active compound can be required to achieve the same inhibition of pathogenic invasion as when the pharmaceutical composition includes the active compound alone. Thus, the therapeutically effective amount of the active compound in a pharmaceutical composition can be inversely proportional to the therapeutically effective amount of antibiotic in the pharmaceutical composition.

In embodiments, the therapeutically effective amount of the antibiotic is sufficient to eliminate extracellular bacteria in a target site of the subject. In one embodiment, the target site includes the mouth of a subject. The target site can be the subject's gingival tissue. In one embodiment, a therapeutically effective amount of the antibiotic is sufficient to achieve a concentration of at least about 50 μg/ml of the antibiotic in the gingival tissue of a subject. The therapeutically effective amount of the antibiotic can be sufficient to achieve a concentration of up to about 1000 μg/ml in the gingival tissue of the subject. The therapeutically effective amount can be about 50 μg/ml, about 100 μg/ml, about 150 μg/ml, about 200 μg/ml, about 250 μg/ml, about 300 μg/ml, about 350 μg/ml, about 400 μg/ml, about 450 μg/ml, or about 500 μg/ml in the gingival tissue of the subject.

In one embodiment of the pharmaceutical composition, the active compound includes GM, and the pharmaceutical composition is configured to be co-administered with an antibiotic. In embodiments, the pharmaceutical composition includes a therapeutically effective amount of GM and a therapeutically effective amount of an antibiotic. In certain embodiments, the antibiotic includes any antibiotic that can eliminate or reduce the incidence of an invasive bacterium in a host cell. In one embodiment, the antibiotic includes any antibiotic that can prevent an invasive bacterium from entering a host cell. The antibiotic can include any antibiotic that can eliminate or reduce the incidence of an oral bacteria in the gingival tissue of a subject. In certain embodiments, the antibiotic includes penicillin, streptomycin, gentamicin, metronidazole, or a combination thereof.

Additional exemplary factors that can influence the therapeutically effective amount include, but are not limited to, the mode of administration, the site of administration, or a combination thereof. The pharmaceutical compositions can be formulated to be provided to the subject in any method known in the art. Exemplary dosage forms include subcutaneous, intravenous, topical, epicutaneous, oral, intraosseous, intramuscular, intranasal, mucosal absorption, and pulmonary. The compositions of the present disclosure may be formulated to be administered only once to the subject or more than once to the subject. Furthermore, when the compositions are administered to the subject more than once, a variety of regimens may be used, such as once per day, once per week, once per month or once per year. The compositions may also be formulated to be administered to the subject more than one time per day. The therapeutically effective amount of the active compound and appropriate dosing regimens may be identified by testing in order to obtain optimal activity, while minimizing any potential side effects. In addition, formulation for co-administration or sequential administration of other agents may be desirable.

The compositions of the present disclosure can be formulated to be administered systemically, such as by intravenous administration, or locally such as by subcutaneous injection or through absorption. The compositions can be formulated for administration by application of a gel, fiber, paste, or cream.

The compositions of the present disclosure can be formulated in a wide variety of dosage forms for administration. For example, the compositions can be in the form of tablets, capsules, sachets, lozenges, troches, pills, powders, granules, elixirs, tinctures, solutions, suspensions, syrups, ointments, creams, pastes, emulsions, or solutions for intravenous administration or injection. Another dosage form includes transdermal administration, such as via a patch or ointment. Further dosage forms include formulations suitable for delivery by nebulizers or metered dose inhalers. Any of the foregoing may be modified to provide for timed release and/or sustained release formulations.

In the present disclosure, the pharmaceutical compositions may further include a pharmaceutically acceptable carrier. Such carriers may include vehicles, adjuvants, surfactants, suspending agents, emulsifying agents, inert fillers, diluents, excipients, wetting agents, binders, lubricants, buffering agents, disintegrating agents and carriers, as well as accessory agents, such as coloring agents and flavoring agents (collectively referred to herein as a carrier). Typically, the pharmaceutically acceptable carrier is chemically inert to the active compounds and has no detrimental side effects or toxicity under the conditions of use. The pharmaceutically acceptable carriers can include polymers and polymer matrices. The nature of the pharmaceutically acceptable carrier may differ depending on the particular dosage form employed and other characteristics of the composition.

For instance, when compositions for oral administration are in a solid form, such as tablets, capsules, sachets, lozenges, troches, pills, powders, or granules, the active compound can be combined with an oral, non-toxic pharmaceutically acceptable inert carrier, such as inert fillers, suitable binders, lubricants, disintegrating agents and accessory agents. Suitable binders include, without limitation, starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include, without limitation, sodium oleate, sodium stearate, magnesium stearate, sodium benzoate, sodium acetate, and the like. Disintegrators include, without limitation, starch, methyl cellulose, agar, bentonite, xanthan gum and the like. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid as well as the other carriers described herein. Lozenge forms can include the active ingredient in a flavor, usually sucrose and acacia or tragacanth, as well as pastilles including the active ingredient in an inert base, such as gelatin and glycerin, or sucrose and acacia, emulsions, and gels containing, in addition to the active ingredient, such carriers as are known in the art. In embodiments, pharmaceutical compositions, which can be used orally, include push-fit capsules including gelatin as well as soft, sealed capsules including gelatin and a plasticizer, such as glycerol or sorbitol. In soft capsules, the pharmaceutical compositions can be dissolved or suspended in suitable liquids, such as fatty oils, liquid paraffin, or liquid polyethylene glycols. In addition, stabilizers can be added. All formulations for oral administration are in dosages suitable for the chosen route of administration.

For buccal administration, the compositions can take the form of tablets or lozenges formulated in conventional manner. In addition, the dentifrice compositions can be utilized for buccal administration.

Dentifrices are widely available to consumers. Indeed, subjects are familiar with the use of dentifrices, and many human subjects apply a dentifrice at least once daily as part of an oral hygiene regime. In addition, dentifrices can be effective for the local delivery of active compounds to gingival tissue or salivary glands.

Exemplary dentifrices can include a paste, a gel, a mouthwash, a powder, and a tooth soap. In some embodiments of the pharmaceutical compositions, the dentifrice is a paste or gel including at least one of an abrasive, a surfactant, a humectant, and a thickener. Exemplary abrasives include, but are not limited to, hydrated silica, dicalcium phosphate dihydrate, calcium carbonate, sodium bicarbonate, calcium pyrophosphate, and alumina. Exemplary surfactants include, but are not limited to, sodium lauryl sulfate, sodium N-lauryl sarcosinate, pluronics, sodium lauryl sulfoacetate. Fluoride represents one exemplary anticaries agent. Exemplary tartar control ingredients include, but are not limited to, tetrasodium pyrophosphate, Gantrez S-70, sodium tripolyphosphate, and methyl vinyl ether/maleic anhydride copolymer. The dentifrice may further include one or more of: water; pH buffers; humectants (to prevent dry-out and increase pleasant mouth feel) such as, glycerin, sorbitol, polypropylene glycol, xylitol, and polyethylene glycol; thickeners such as silica thickeners, sodium aluminum silicates, and clays; gums such as sodium carboxymethyl cellulose, cellulose ethers, xanthan gum, carrageenans, sodium alginate, and carbopols; antibacterial agents; flavoring agents such as, water-insoluble essential oils; sweetening agents such as, saccharin, dextrose, levulose, cyclamate, aspartate; coloring agents; and binders to provide consistency and shape.

For oral administration by mouthwash, the active compound can be combined with one or more of: water and alcohol (such as ethyl alcohol). The mouthwash can further include one or more of: surfactants, tartar control ingredients, anticaries agents, buffers, humectants, antibacterial agents, flavoring agents, and coloring agents as described herein.

In a specific embodiment, the dentifrice is a powder including any of the abrasives described herein. The powder can further include any of the dry components provided herein as suitable in a toothpaste. In another specific embodiment, the dentifrice is a tooth soap including one or more of oil and water. The oil can be any that is known to be suitable in a tooth soap, such as olive oil, coconut oil, an essential oil, a peppermint oil, or any combination thereof.

The pharmaceutical composition can be a chewing gum. The gum can include the active compound and a gum, such as butadiene-based synthetic rubber, birch bark tar, chicle, mastic gum, spruce gum, paraffin wax, tolu resin, styrene-butadiene rubber, isobutylene, isoprene copolymer, and petroleum wax. The gum can be present at a concentration sufficient to confer the requisite texture to ensure chewiness of the chewing gum, as could be formulated by one skilled in the art. Flavorings can be added, including those listed above.

In addition to dentifrice and gum forms of the composition, oral liquid forms, such as tinctures, solutions, suspensions, elixirs and syrups, of the compounds of the present disclosure can be dissolved in diluents, such as water, saline, or alcohols. Furthermore, the oral liquid forms can include suitably flavored suspending or dispersing agents such as synthetic and natural gums, for example, tragacanth, acacia, methylcellulose and the like. Moreover, when desired or necessary, suitable coloring agents or other accessory agents can also be incorporated into the mixture. Other dispersing agents that can be employed include glycerin and the like.

For administration by nasal inhalation, the pharmaceutical compositions can be delivered in the form of an aerosol spray presentation from a pressurized pack or a nebulizer with the use of a suitable propellant, for example, dichlorodifluoromethane, trichlorofluoromethane, dichloro-tetrafluoroethane, or carbon dioxide. In the case of a pressurized aerosol, the dosage unit can be determined by providing a valve to deliver a metered amount. Capsules and cartridges of, for example, gelatin for use in a dispenser can be formulated containing a powder mix of the compound and a suitable powder base such as lactose or starch.

The pharmaceutical compositions described herein can be formulated for parenteral administration, for instance, by bolus injection or continuous infusion. Formulations for injection can be presented in unit dosage form, for example, in ampoules or in multidose containers with optionally, an added preservative. The compositions can be suspensions, solutions, or emulsions in oily or aqueous vehicles, and can contain formulatory agents such as suspending, stabilizing and/or dispersing agents. Pharmaceutical compositions for parenteral administration can include aqueous solutions of the pharmaceutical compositions in water-soluble form. Additionally, suspensions of the pharmaceutical compositions can be prepared as appropriate oil or water-based injection suspensions. Suitable lipophilic solvents or vehicles include fatty oils or synthetic fatty acid esters such as ethyl oleate, triglycerides or liposomes. Aqueous injection suspensions can contain substances, which increase the viscosity of the suspension, such as sodium carboxymethyl cellulose, sorbitol or dextran. Optionally, the suspension can also contain suitable stabilizers or agents to increase the solubility of the pharmaceutical compositions to allow for the preparation of concentrated solutions. Formulations suitable for parenteral administration include aqueous and non-aqueous, isotonic sterile injection solutions, which can contain antioxidants, buffers, bacteriostats, and solutes that render the formulation isotonic with the blood of the patient, and aqueous and non-aqueous sterile suspensions that can include suspending agents, solubilizers, thickening agents, stabilizers, and preservatives. The compound(s) may be administered in a physiologically acceptable diluent, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethylene glycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as a soap, an oil or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in certain embodiments of the pharmaceutical compositions include petroleum, animal, vegetable, or synthetic oils. Oils can be useful in certain pharmaceutical composition embodiments in parenteral formulations, in the dentifrice formulations, or a combination thereof. Specific, non-limiting examples of oils include peanut, soybean, sesame, cottonseed, corn, olive, petrolatum, and mineral. Suitable fatty acids for use in certain pharmaceutical compositions include polyethylene sorbitan fatty acid esters, such as sorbitan monooleate and the high molecular weight adducts of ethylene oxide with a hydrophobic base, formed by the condensation of propylene oxide with propylene glycol, oleic acid, stearic acid, and isostearic acid. Ethyl oleate and isopropyl myristate are non-limiting examples of suitable fatty acid esters. Suitable soaps for use in parenteral formulations include fatty alkali metal, ammonium, and triethanolamine salts, and suitable detergents include: (a) cationic detergents such as, for example, dimethyldialkylammonium halides, and alkylpyridinium halides; (b) anionic detergents such as, for example, alkyl, aryl, and olefin sulfonates, alkyl, olefin, ether, and monoglyceride sulfates, and sulfosuccinates; (c) nonionic detergents such as, for example, fatty amine oxides, fatty acid alkanolamides, and polyoxyethylene polypropylene copolymers; (d) amphoteric detergents such as, for example, alkylbeta-aminopropionates, and 2-alkylimidazoline quaternary ammonium salts; and (e) mixtures thereof.

Suitable preservatives and buffers can be used in certain formulations of pharmaceutical compositions. In order to minimize or eliminate irritation at the site of injection, such compositions may contain one or more nonionic surfactants having a hydrophile-lipophile balance (HLB) of from about 12 to about 17.

Topical dosage forms, such as ointments, creams, pastes, and emulsions, containing the active compound, can be admixed with one or more carrier materials well known in the art, such as, for example, alcohols, aloe vera gel, allantoin, glycerine, vitamin A and E oils, mineral oil, PPG2 myristyl propionate, and the like, to form alcoholic solutions, topical cleansers, cleansing creams, skin gels, skin lotions, and shampoos in cream or gel formulations. Inclusion of a skin exfoliant or dermal abrasive preparation may also be used. Such topical preparations can be applied to a patch, bandage or dressing for transdermal delivery, or can be applied to a bandage or dressing for delivery directly to the site of a wound or cutaneous injury.

Alternatively, the pharmaceutical compositions can be in powder form for constitution with a suitable vehicle, for instance, sterile, pyrogen-free water-based solution, before use.

The preparations can also be formulated in rectal compositions such as suppositories or retention enemas, using, for example, conventional suppository bases such as cocoa butter or other glycerides.

The active compounds of the present disclosure can also be formulated to be administered in the form of liposome delivery systems, such as small unilamellar vesicles, large unilamellar vesicles and antiemtrics. Liposomes can be formed from a variety of phospholipids, such as cholesterol, stearylamine, or phosphatidylcholines. Such liposomes may also contain monoclonal antibodies to direct delivery of the liposome to a particular cell type or group of cell types.

The compositions of the present disclosure can further include agents which improve the solubility, half-life, absorption, etc. of the active compound. Furthermore, the compositions of the present disclosure can further include agents that attenuate undesirable side effects and/or decrease the toxicity of the active compound. Examples of such agents are described in a variety of texts, such as Remington: The Science and Practice of Pharmacy (20^{th} Ed., Lippincott, Williams & Wilkins, Daniel Limmer, editor).

Certain pharmaceutical composition embodiments include active compounds coupled with soluble polymers as targetable drug carriers. Such polymers can include polyvinyl-pyrrolidone, pyran copolymer, polyhydroxypropylmethacryl-amidephenol, polyhydroxyethylaspartamidephenol, or polyethyleneoxidepolylysine substituted with palmitoyl residues. Furthermore, the active compounds of the present invention can be coupled to a class of biodegradable polymers useful in achieving controlled release of a drug, for example, polylactic acid, polyepsilon caprolactone, polyhydroxy butyric acid, polyorthoesters, polyacetals, polydihydro-pyrans, polycyanoacrylates and cross-linked or amphipathic block copolymers of hydrogels.

### D. Methods of Treatment and Prevention

The present disclosure provides a method of treatment or prevention of a disease or medical condition associated with or characterized by periodontitis in a subject in need thereof, the method including administering any of the active compounds or pharmaceutical compositions disclosed herein to the subject in a therapeutically effective amount. One specific example of such an active compound is GM.

Exemplary conditions associated with or characterized by periodontitis include periodontitis, atherosclerosis, human immunodeficiency virus (HIV) disease, tooth loss, coronary artery disease, stroke, premature birth, low birth weight, poorly controlled diabetes, respiratory problems, rheumatoid arthritis, and asthma. Furthermore, some medical treatments are associated with xerostomia, which can lead to periodontitis. Such medical treatments include, but are not limited to anticholinergic drugs, drugs with sympathomimetic actions, local radiation, and chemotherapy. Anticholinergic drugs that may give rise to xerostomia include, but are not limited to atropine and analogs (antimuscarinics), tricyclic antidepressants, serotonin reuptake inhibitors, antihistamines, antiemetics, and antipsychotics. Drugs with sympathomimetic actions associated with xerostomia include, but are not limited to decongestants, bronchodilators, appetite suppressants, and amphetamines. Other treatments that are also associated with xerostomia include, but are not limited to lithium, omeprazole, oxybutynin, disopyramide, dideoxyinosine, didanosine, diuretics, and protease inhibitors. In some embodiments of the method the subject has undergone or is currently undergoing a medical treatment associated with xerostomia, including but not limited to any of the treatments described above. A further embodiment of the method includes co-administering a treatment associated with at least one of xerostomia and periodontitis to the subject, including but not limited to any of the treatments described herein.

The method of treatment or prevention includes administering to the subject the active compound in an amount sufficient to treat or prevent periodontitis or a periodontitis-related condition or symptom. The method can further include identifying a subject in need of such treatment or prevention. The methods can include administering a therapeutically effective amount of the active compound to the subject.

As disclosed herein, the therapeutically effective amount can vary according to a variety of factors. Exemplary factors include the subject's condition, weight, sex and age. For example, some embodiments of the method include administration of up to the median lethal dose (LD₅₀) of the active compound. The LD₅₀ can be ascertained using standard toxicological methods, or by reference to past studies.

The method can include delivering a desired concentration of the active compound to the gingival tissue of the subject. In some embodiments of the method, the desired concentration is a therapeutically effective amount.

If, after the administration of the active compound, the subject still has periodontitis or a periodontitis-related condition or symptom, or is at risk for the same, then an optional step of the method can be to continue administration of the active compound or pharmaceutical composition for an extended treatment period. In embodiments, the extended treatment period is an amount of time that is sufficient to substantially eliminate periodontitis or the periodontitis-related condition. The extended treatment period can be up to about 6 months. The extended treatment period can be as little as about one day. In embodiments, the extended treatment period is about one week, about two weeks, about three weeks, about four weeks, about five weeks, about six weeks, about seven weeks, about eight weeks, about nine weeks, or about ten weeks. The extended treatment period can be less than one week. In embodiments, the extended treatment period is about one day, about two days, about three days, about four days, about five days, about six days, about seven days, about eight days, about nine days, about ten days, about eleven days, about twelve days, about thirteen days, or about fourteen days.

In one embodiment, the method of treatment or prevention includes administering the active compound or pharmaceutical composition to the subject along with an antibiotic or a combination of two or more antibiotics. In one embodiment, at least one antibiotic and the active compound are provided to the subject in a single pharmaceutical composition. In an alternate embodiment, the at least one antibiotic and the active compound are administered as separate pharmaceutical compositions or formulations. One embodiment includes administration of a pharmaceutical composition to the subject that includes a therapeutically effective amount of an active compound and a therapeutically effective amount of an antibiotic. In certain embodiments, co-administration of an antibiotic with the active compound generally increases the therapeutic effectiveness of the active compound. Co-administering an antibiotic with a pharmaceutical composition that includes an active compound can produce a synergistic effect that allows for more effective treatment or prevention of the disease or medical condition associated with periodontitis. In some respects, when the pharmaceutical composition includes an antibiotic, a lower concentration of active compound achieves the same treatment or prevention effects in the subject as when the pharmaceutical composition includes the active compound alone. Thus, the therapeutically effective amount of the active compound administered to the subject can be inversely proportional to the therapeutically effective amount of antibiotic administered to the subject.

In one embodiment, the method of treatment or prevention of a disease or medical condition associated with or characterized by periodontitis in a subject in need thereof, includes administering the active compound to the gingival tissue of the subject. In such embodiments, delivery the active compound to the subject's gingival tissue prevents intracellular invasion by at least one periodontal pathogen, such as P. gingivalis. In various embodiments, targeted delivery of the active compound to the gingival tissue can prevent unwanted effects on other tissues or organs. In an embodiment, the method includes administering the active compound locally to the subject's mouth. A specific embodiment includes administering the active compound locally to the subject's mouth in which the active compound is administered as a dentifrice or gum. A dentifrice or gum containing active compound can be useful to treat or prevent periodontitis or a periodontitis-related condition or symptom. Such formulations can further include additives to improve overall oral health (for example, fluoride). In embodiments, the method includes administration of the dentifrice or gum that includes the active compound as a part of the subject's established oral hygiene routine. Such a treatment method can be particularly useful for human subjects who already utilize similar formulations for routine oral hygiene.

In an embodiment, the method include administering GM to the subject until Pg is eliminated from the oral cavity of the subject.

The methods of administration of the composition described herein to the subject may vary. In one embodiment, the active compound or pharmaceutical composition is administered to the subject orally. For example, the active compound or pharmaceutical composition can be encapsulated or tableted for a solid oral dosage form. In one embodiment, the active compound or pharmaceutical composition is administered in the form of a pill, tablet, capsule, or gel capsule.

The active compound or pharmaceutical composition can be provided in a liquid, gel, or powder form. For example, the active compound or pharmaceutical composition can be in the form of a powder suitable for mixing with water or other liquids. These formulations can be added into a beverage or can be provided as an ingredient premixed in a beverage. The active compound or pharmaceutical composition can be administered as an elixir or as a solution formulation. In another embodiment, the active compound or pharmaceutical composition is administered in the form of a nutritional or dietary supplement. In still another embodiment, the active compound or pharmaceutical composition is administered in the form of a functional food, for example, a protein bar. Moreover, the compositions of the invention can be administered intravenously.

Suitable exemplary routes of administration include oral, rectal, transmucosal, transnasal, intestinal, or parenteral delivery, including intramuscular, subcutaneous, and intramedullary injections as well as intrathecal, direct intraventricular, intravenous, intraperitoneal, intranasal, or intraocular injections. Alternately, the preparation can be administered in a local rather than systemic manner.

Pharmaceutical compositions can be manufactured by processes well known in the art, for example, by means of conventional mixing, dissolving, granulating, dragee-making, levigating, emulsifying, encapsulating, entrapping or lyophilizing processes. Pharmaceutical compositions for use in the disclosed methods thus can be formulated in a conventional manner using one or more physiologically acceptable carriers including excipients and auxiliaries, which facilitate processing of the pharmaceutical compositions into preparations that can be used pharmaceutically. The method of formulation can be dependent upon the anticipated route of administration for the pharmaceutical composition.

For instance, when the active compound is to be injected, the pharmaceutical composition can be formulated in aqueous solutions, preferably in physiologically compatible buffers, Exemplary physiologically compatible buffers include, but are not limited to, Hank's solution, Ringer's solution, or a physiological salt buffer. For transmucosal administration, certain penetrants that are appropriate for transport of the pharmaceutical composition across the applicable barrier to be permeated are used in the formulation. Such penetrants are generally known in the art.

For oral administration, the compounds can be formulated readily by combining the pharmaceutical compositions with pharmaceutically acceptable carriers. Such carriers enable the compounds to be formulated as tablets, pills, dragees, capsules, liquids, gels, syrups, slurries, suspensions, and the like, for oral ingestion by a subject or patient. Pharmacological preparations for oral use can be made using a solid excipient, optionally grinding the resulting mixture, and processing the mixture of granules, after adding suitable auxiliaries if desired, to obtain tablets or dragee cores. Suitable excipients can include fillers. Exemplary, non-limiting fillers include, but are not limited to, sugars (including lactose, sucrose, mannitol, or sorbitol), cellulose preparations such as, for example, maize starch, wheat starch, rice starch, potato starch, gelatin, gum tragacanth, methyl cellulose, hydroxypropylmethyl-cellulose, sodium carbomethylcellulose, physiologically acceptable polymers such as polyvinylpyrrolidone (PVP), or combinations thereof. In certain embodiments, disintegrating agents can be added, such as cross-linked polyvinyl pyrrolidone, agar, or alginic acid or a salt thereof such as sodium alginate.

Dragee cores can include suitable coatings. For this purpose, concentrated sugar solutions can be used which can optionally contain gum arabic, talc, polyvinyl pyrrolidone, carbopol gel, polyethylene glycol, titanium dioxide, lacquer solutions, suitable organic solvents or solvent mixtures, or any combination thereof. Dyestuffs or pigments can be added to the tablets or dragee coatings for identification or to characterize different combinations of pharmaceutical composition doses.

### E. Methods of Prevention of Invasion of a Host Cell

A method of reducing the likelihood of the invasion of a host cell by an invasive pathogen is also provided herein. In embodiments, the method includes contacting the host cell with an effective concentration of any of the active compounds discussed above. Contact may occur in vitro or in vivo. In various embodiments, the methods disclosed herein can be applied to prevent intracellular invasion of bacteria, fungi, viruses, protists, or a combination thereof. Non-limiting examples of bacteria include P. gingivalis, Bartonella henselae, Francisella tularensis, Listeria monocytogenes, Salmonella typhi, Brucella spp., Legionella spp., Mycobacterium spp., Nocardia spp., Rhodococcus equi, Yersinia spp., Neisseria meningitidis, Chlamydia spp., Rickettsia spp., and Coxiella spp. Examples of intracellular parasitic fungi include, but are not limited to, Histoplasma capsulatum, Cryptococcus neoformans, and Pneumocystis jirovecii. Protozoal examples include apicomplexans (e.g., Babesia spp., Plasmodium spp., Cryptosporidium parvum, Cyclospora cayetanensis, Isospora belli, and Toxoplasma gondii) and trypanosomatids. In some embodiments of the method, the intracellular bacteria is an oral pathogen, regardless of its taxon.

The effective concentration is any concentration at which the active compound exerts an inhibitory effect on cellular invasion by the invasive pathogen. In one embodiment, the effective concentration is the therapeutically effective amount of the active compound. The effective concentration can be an amount of the active compound that achieves a desired concentration of the active compound in the gingival tissue of the subject. In some embodiments, the effective concentration of the active compound is sufficient to achieve a concentration of at least about 1 nM active compound in the gingival tissue of a subject. In some embodiments, the effective concentration of the active compound is sufficient to achieve a concentration of up to about 10 μM in the gingival tissue of a subject. In one embodiment, the effective concentration of the active compound is an amount sufficient to achieve a concentration of at least about 20 nM active compound in the gingival tissue of a subject. The effective concentration of active compound can be sufficient to achieve a concentration of between about 5 nM and 1 μM active compound in the gingival tissue of a subject. In specific embodiments, the effective concentration of active compound is sufficient to achieve a concentration of at least one of about 10 nM, about 20 nM, about 30 nM, about 40 nM, about 50 nM, about 60 nM, about 70 nM, about 80 nM, about 90 nM, and about 100 nM in the gingival tissue of a subject. In one specific embodiment the effective concentration of GM is sufficient to achieve a concentration of at least about 5 nM, at least about 10 nM, at least about 15 nM, at least about 20 nM, at least about 25 nM, at least about 30 nM, at least about 35 nM, or at least about 40 nM in the gingival tissue.

### F. Methods of Screening

A method of screening a candidate factor for activity in treating or preventing periodontitis or a periodontitis-related condition or symptom is provided, the method including exposing a plurality of host cells to the candidate factor in the presence of an intracellular bacteria and measuring the rate of infection of the plurality of host cells by the intracellular bacteria. The bacteria and the host cells can be any that are taught herein.

The candidate factor may be any type of factor, including a physical factor, a chemical factor, a genetic factor, a biochemical factor, or an ecological factor. Examples of physical factors include temperature, oxidation potential, pressure, and radiation. Examples of chemical factors include osmotic potential, concentration of inorganic compounds, organic compounds, pH, and salinity. Examples of biochemical factors include a nutrient concentration, a prion, and a toxin concentration. Examples of ecological factors include population density, intrinsic rate of growth, carrying capacity, predation, parasitism, and growth rate. Examples of genetic factors include a genotype, a mutation, an episome, a transposable genetic element, a virus, and a viroid. However, as the purpose of the assay is to identify putative treatment or prevention factors that were previously unknown, it should be emphasized that the factor may be any factor to which the cell can be subjected.

The method of measuring the rate of infection may be any known in the art. In a specific embodiment of the method, the rate of infection is measured by staining a mixture of host cells and bacteria and examining the stained mixture microscopically. Fluorescent dyes can be used for this purpose to enhance the signal when viewed by epifluorescent microscopy. The use of fluorescent dyes also allows fluorometry to be used to measure the number of intracellular bacteria present (for example, after separation of host cells from free bacteria). In a specific embodiment, the rate of infection is measured by permeabilizing the host cells, immunostaining the sample with a fluorescent probe, acquiring confocal microscopic images of the immuno-stained sample, enumerating the total number of host cells by direct count, and enumerating the number of host cells harboring intracellular bacteria by direct count. Such direct counting methods can involve counting cells in a set number of microscopic fields and extrapolating the results to the entire sample. However, any approach to the direct count may be employed.

Some embodiments of the method are intended to screen specifically for factors that decrease the rate of pathogenic invasion of the host cell. In such embodiments the factor can be a non-antibiotic factor (i.e., a factor which is not necessarily toxic to the bacteria). Such embodiments of the method may also involve counting the total number of bacteria after exposure to the factor, to differentiate between factors that have antibiotic activity and factors that have anti-invasive activity.

In some embodiments of the method, the host cell is a gingival tissue cell. The gingival tissue cell can be an oral epithelial cell or a gingival fibroblast. Exemplary gingival tissue cells include those from various mammalian species, including humans and domesticated animals. In a further specific embodiment of the method, the host cell is an oral keratinocyte, such as an oral keratinocyte from a human or a domesticated animal. Moreover, although the examples provided herein relate to inhibition of Pg, one of skill in the art understands that Pg can be used as a model for developing therapeutics against other invasive pathogens.

Well known statistical methods can be used to detect a decrease in infection rate of a sample exposed to the factor relative to a benchmark value. The benchmark value can be established using a parallel control study or can be obtained from the literature describing previous studies. However, in some instances such statistical methods may not be necessary due to obvious effects of the factor.

### G. EXAMPLES

### 1. Example 1: Activity of Gnidimacrin (GM) and its Derivatives and Analogs Against Human Cell Invasion by Bacteria

The microbiome plays a vital role in human health and involves digestion and immune responses of our bodies, and it is also associated with infectious and dysbiotic diseases^{1,2}. Modulation of the microbiota including elimination of keystone pathogens has been considered as a strategy for the therapeutics to prevent or treat microbiome-associated diseases. Porphyromonas gingivalis (Pg) is considered a keystone pathogen of periodontitis able to transform a commensal microbial community into a pathogenic one that induces a breakdown of periodontal tissue-microbe homeostasis and leads to uncontrolled inflammation and tissue destruction. Inhibition and elimination of Pg is, therefore, an attractive strategy to prevent and treat chronic periodontitis. However, despite great effort, development of therapeutic agents selectively targeting Pg, especially intracellular Pg, is limited. Difficulties in eliminating Pg from the oral cavity are due to Pg's ability to invade host cells and the drug-resistant nature of dental plaques, which facilitate the organism in evading host defense systems and antibiotics. Pg may multiply in the host cells and in extracellular matrix, and as a result, create reservoirs for a persistent infection. Therefore, identification of inhibitors targeting Pg in host cells is a rational approach to block progress of periodontitis.

Bacterial invasion is a process in which bacteria actively enter non-phagocytic cells, such as epithelial cells. In general, invasive bacteria express surface invasins that interact with their corresponding receptors on the surface of non-phagocytic cells. As a result, a cascade of signaling events in the host cells is triggered, including protein phosphorylation, cytoskeleton rearrangements, membrane extension, and bacterial engulfment. After internalization, bacteria may survive and multiply in the cytosol as well as spread to other host cells. The ability to invade host cells empowers bacteria to establish a successful infection by avoiding immune responses and antibiotic treatment. Intracellular bacteria also are known to contribute to persistence of chronic infections by serving as opportunity bacteria³. Although the mechanisms by which bacteria entering host cells are well documented, studies on the inhibition of bacterial invasion are limited.

One bacterial invasion example is Porphyromonas gingivalis (Pg), a keystone pathogen of chronic periodontitis. The disease is the 6^{th} most common infection worldwide+, with an estimated 5-20% of the population suffering from chronic periodontitis^{5,6}. Pg possesses a vast array of effector molecules and systems that enable its survival in the oral cavity⁷⁻¹⁰. One of Pg's important features is its ability to invade and survive within host cells such as fibroblasts, epithelial, and endothelial cells¹¹. Several membrane proteins involved in Pg attachment to, and invasion of, host cells have been identified, including FimA, hemagglutinin A, and heat-stress protein¹²⁻¹⁵. Pg strains with invasion deficiency exhibit an approximate 10 times decrease in invasive activity¹⁶ and could not induce the alveolar bone loss in a gnotobiotic rat that was observed in the wild Pg strain¹⁷. Apparently, invasive ability is important in establishing Pg infection in the oral cavity, and inhibition of Pg invasion may provide a new therapeutic strategy to prevent the establishment of a Pg reservoir. Moreover, Pg can be used as a model of an invasive bacterial pathogen and has served as a paradigm for developing therapeutics against other invasive bacterial pathogens. Although the role of bacterial invasion in its pathogenicity has been investigated by microbiologists, studies on bacterial invasion inhibitors are limited.

As presently disclosed herein, a natural compound, gnidimacrin (GM) effectively blocks Pg and its vesicles from invasion into primary human oral keratinocytes (HOKs) at a low nanomole concentration (20 nM). GM also does not affect host cell viability. These findings suggest that potent inhibitory activity and low cytotoxicity make GM an ideal candidate for anti-Pg drug development.

Treatment of chronic periodontitis. Conventional therapies for periodontitis, including scaling and root planing, are still the gold standard treatments for chronic periodontitis. However, the conventional treatments cannot prevent the re-emergence of a pathogenic microbial community, as previous studies have revealed high levels of Pg at the periodontal sites of patients with refractory periodontitis^{18,19}. Systemic or local administration of antibiotics may be used adjunctively to combat severe and refractory manifestations of the disease²⁰⁻²². However, recommended dosing for the treatment could not kill intracellular Pg which may proliferate intracellularly and spread to adjacent cells ²³. In addition, there are increasing public health concerns regarding the use of antibiotics, including disruption of the symbiotic or mutualistic relationships between host and commensal microbiota²⁴, and emerging oral bacterial resistance to antibiotics. Further, these treatment strategies are often effective only temporarily, and recurrence of the disease is common^{25,26}, possibly due to the re-emergence of pathogens²⁷. Therefore, novel therapeutic agents that can specifically eliminate intracellular Pg would be attractive, and combination of Pg invasion inhibitor and antibiotics treatments would likely enhance chance to eliminate Pg from the oral cavity. Therefore, this example focuses on the inhibitory mechanism of GM against Pg invasion and development of more practicable and efficient candidates for periodontal therapies.

Scientific Premise. The present example is built on evidence that current therapeutic options for periodontitis are only partially successful, and that lifetime periodontal treatment and maintenance are required and costly. Intracellular Pg can survive periodontal mechanical therapies and resists antibiotic treatments. Selective targeting keystone periodontal pathogens such as Pg will provide an effective supplemental treatment for fully eliminating Pg in host cells. Equally important, identification of the anti-invasive compounds may also open new avenues for the prevention and treatment of infectious diseases caused by other invasive bacteria.

As disclosed herein, GM represents a potential therapeutic mechanism for efficiently eliminating intracellular Pg. Conventional therapies for periodontitis are still the gold standard treatments. However, they cannot prevent the re-emergence of a pathogenic microbial community, as evidenced by the presence of high levels of Pg at periodontal sites of patients with refractory periodontitis^{18,19}. Currently, ARESTIN® (minocycline HCl) is used as an adjunctive treatment in clinic to ensure the elimination of periodontal pocket bacteria. However, the concerns for such a treatment include its potential to induce antibiotic resistance, its nonspecific action, its inability to eliminate intracellular bacteria. Ultimately, traditional antibiotic treatment may induce overgrowth of periodontitis-associated bacteria after wiping out most of the oral bacteria. The invasion and colonization abilities of Pg pose challenges for conventional periodontitis treatments. GM, as an efficient inhibitor of Pg invasion with low toxicity and high-water solubility, can be an ideal anti-Pg agent. One advantage of GM disclosed herein is its potential for use in developing of agents for eliminating intracellular Pg, such as by blocking the bacterial entry and re-entry into host cells.

### Preliminary Results

Gnidimacrin (GM) is a diterpene compound, which can be isolated from Stellera chamaejasme.

Inhibition of Pg cells invasion by GM. The ability of Pg to invade oral epithelial cells was determined using an antibiotic protection assay as described²⁸. Human primary oral keratinocytes (HOKs, 5×10⁴ cells, ScienCell® Research Laboratories) were seeded in a 24-well plate. After incubating with Pg 33277 for 1 h, HOKs were continually cultured for another 4 h in the presence of antibiotics (gentamicin (300 μg/ml) and metronidazole (200 μg/ml)) to eliminate extracellular bacteria. The HOKs were then lysed, and the internalized bacteria were plated on TSB blood agar plates. The plates were incubated anaerobically at 37° C. for 7 days, and CFUs of Pg were enumerated. As shown in FIG. 2A, GM significantly reduced invasion of Pg cells into HOKs in a dose-dependent manner. In the presence of 40 nM GM, more than 95% of Pg were inhibited from entry into HOKs. In addition, the role of GM in Pg re-entry was tested. After exposure to Pg cells for 1 h and then antibiotic treatment for 4 h, HOKs were continuously cultured for 24 h in the presence of GM (20 nM), antibiotics, or GM and antibiotics. Intracellular Pg was determined as described²⁸. As expected, intracellular Pg cells moved in and out of the HOKs. Once outside of the HOKs, Pg re-entry was block by GM, and the extracellular Pg cells were killed by antibiotics (FIG. 2B). As can be seen, the combination of GM and antibiotics showed synergistic effects and removed about 90% of intracellular Pg.

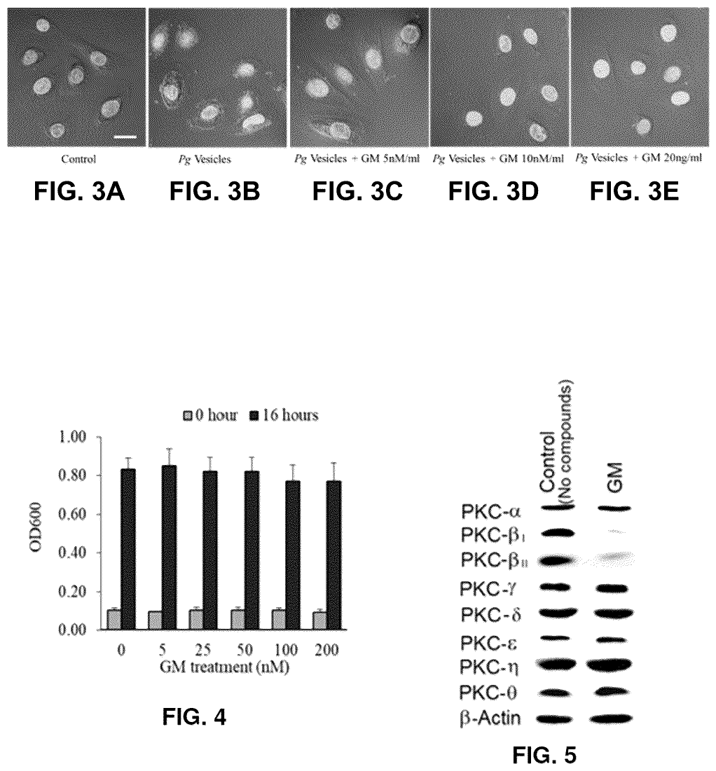

Inhibitory activity and efficiency of GM on Pg vesicle invasion. Pg vesicles were previously reported as being able to efficiently invade oral epithelial cells²⁹. GM was recently tested for its role in Pg vesicle invasion using confocal microscopy. GM inhibited Pg vesicle invasion of HOKs in a dose-dependent manner (FIGS. 3A-3E). GM almost blocked the vesicle invasion at 20 nM. Previously, it was reported that two compounds, Alopl and dynasore, also inhibited the vesicle invasion at 30 μM30. These results indicate that GM is a more potent and efficient inhibitor of Pg vesicles invasion than previously studied compounds.

Effects of GM on bacterial growth and host cell viability. The role of GM on the growth of Pg 33277 in Trypticase soy broth (TSB) was examined. Bacterial growth in TSB was determined by their optical density at 600 nm. As shown in FIG. 4, growth rates of Pg were not significantly affected in the presence of GM up to 200 μM as compared to growth without GM.

GM selectively activates (protein kinase C-β) PKC-β. It has been previously shown that GM is PKC-β activator with potent dichotomous activity against HIV-1 replication^{31,32}. In brief, in order to determine whether GM is a selective PKC agonist, anti-CD3/CD28 (1 μg/ml) activated human primary CD+ cells were cultured in the presence or absence of GM (1 nM) for 12 hours. The cytosolic fraction of the cells was analyzed with 10% SDS PAGE followed by Western blot analysis using a panel of monoclonal antibodies against PKC isozymes (Santa Cruz Biotech). PKC activation is expected to decrease PKC level due to translocation of the enzyme from cytosol to membrane. As shown in FIG. 5, GM significantly reduced the cytosolic PKC-β 1 and 2 levels without affecting six other PKC isozymes. This result suggests that GM is a selective PKC-β agonist. Without being bound by any theory, PKC-β selective activity will also be observed in HOKs.

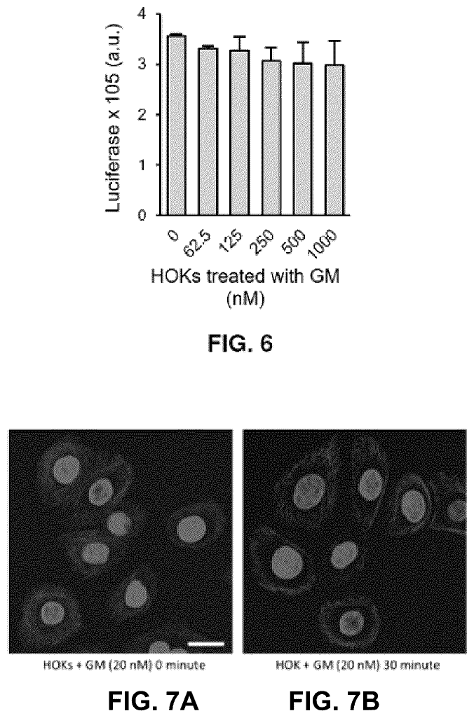

To examine potential cytotoxic effects of GM, HOKs were cultured in the presence of a range of GM from 0 to 1,000 nM for 48 h and the number of viable cells in the culture was determined using Promega™ CellTiter-Glo™. As shown in FIG. 6, GM exposure at concentrations as high as luM (approximately 50-times greater than the concentration that was able to inhibit Pg invasion) for 48 h has no impact upon cell viability of HOKs, when compared to the cells without GM exposure.

Effect of GM in cytoskeleton of oral epithelial cells. Cytoskeletal dynamics are involved in bacterial invasion. To study whether GM affects cytoskeletal structure of epithelial cells, HOKs were treated with GM and then immune-stained with a Tubulin or Actin mono-antibodies, and the cells were observed under a confocal microscope. Microtubule rearrangement in HOKs and formation of a microtubule shell under the cell membrane were found after 30 min of treatment with GM (FIG. 7B). However, significant actin remodeling was not observed after the treatment (Data not shown).

### In Summary:

1. Gnidimacrin and its derivatives and analogs inhibit human cell invasion by P. gingivalis. Without being bound by any theory, Gnidimacrin may also prevent invasion of other oral bacteria into human cells including, but not limited to any one or more of the following: Treponema denticola, Fusobacterium micleatum, and Tannerella forsythia.
2. Gnidimacrin can be used alone or in combination with antibiotics to eliminate P. gingivalis from the oral cavity.
3. Gnidimacrin represents a prospective therapy for periodontitis and can open new avenues for the prevention and treatment of infectious diseases caused by invasive bacteria.

### REFERENCES CITED IN THIS EXAMPLE

1. Libertucci, J. & Young, V. B. The role of the microbiota in infectious diseases. Nature microbiology 4, 35-45 (2019).
2. Hajishengallis, G. & Lamont, R. J. Polymicrobial communities in periodontal disease: Their quasi-organismal nature and dialogue with the host. Periodontology 2000 (2021).
3. Cornejo, E., Schlaermann, P. & Mukherjee, S. How to rewire the host cell: A home improvement guide for intracellular bacteria. The Journal of cell biology 216, 3931-3948 (2017).
4. Kassebaum, N.J., et al. Global burden of severe periodontitis in 1990-2010: a systematic review and meta-regression. Journal of dental research 93, 1045-1053 (2014).
5. Burt, B., Research, S. & Therapy Committee of the American Academy of, P. Position paper: epidemiology of periodontal diseases. Journal of periodontology 76, 1406-1419 (2005).
6. Petersen, P. E. & Ogawa, H. The global burden of periodontal disease: towards integration with chronic disease prevention and control. Periodontology 2000 60, 15-39 (2012).
7. Hajishengallis, G., McIntosh, M. L., Nishiyama, S. I. & Yoshimura, F. Mechanism and implications of CXCR4-mediated integrin activation by Porphyromonas gingivalis. Molecular oral microbiology 28, 239-249 (2013).
8. Lamont, R. J. & Jenkinson, H. F. Life below the gum line: pathogenic mechanisms of Porphyromonas gingivalis. Microbiology and molecular biology reviews: MMBR 62, 1244-1263 (1998).
9. Lamont, R. J. & Jenkinson, H. F. Subgingival colonization by Porphyromonas gingivalis. Oral microbiology and immunology 15, 341-349 (2000).
10. Xie, H. Biogenesis and function of Porphyromonas gingivalis outer membrane vesicles. Future microbiology 10, 1517-1527 (2015).
11. Tribble, G. D. & Lamont, R. J. Bacterial invasion of epithelial cells and spreading in periodontal tissue. Periodontology 2000 52, 68-83 (2010).
12. Belanger, M., Kozarov, E., Song, H., Whitlock, J. & Progulske-Fox, A. Both the unique and repeat regions of the Porphyromonas gingivalis hemagglutin A are involved in adhesion and invasion of host cells. Anaerobe 18, 128-134 (2012).
13. Zhang, W., Ju, J., Rigney, T. & Tribble, G. D. Fimbriae of Porphyromonas gingivalis are important for initial invasion of osteoblasts, but not for inhibition of their differentiation and mineralization. Journal of periodontology 82, 909-916 (2011).
14. Yilmaz, O., Watanabe, K. & Lamont, R. J. Involvement of integrins in fimbriae-mediated binding and invasion by Porphyromonas gingivalis. Cellular microbiology 4, 305-314 (2002).
15. Yuan, L., Rodrigues, P. H., Belanger, M., Dunn, W. A., Jr. & Progulske-Fox, A. Porphyromonas gingivalis htrA is involved in cellular invasion and in vivo survival. Microbiology 154, 1161-1169 (2008).
16. Mantri, C. K., Chen, C., Dong, X., Goodwin, J. S. & Xie, H. Porphyromonas gingivalis-mediated Epithelial Cell Entry of HIV-1. Journal of dental research 93, 794-800 (2014).
17. Malek, R., et al. Inactivation of the Porphyromonas gingivalis fimA gene blocks periodontal damage in gnotobiotic rats. Journal of bacteriology 176, 1052-1059 (1994).
18. Colombo, A. P., et al. Impact of periodontal therapy on the subgingival microbiota of severe periodontitis: comparison between good responders and individuals with refractory periodontitis using the human oral microbe identification microarray. Journal of periodontology 83, 1279-1287 (2012).
19. Colombo, A. P., et al. Comparisons of subgingival microbial profiles of refractory periodontitis, severe periodontitis, and periodontal health using the human oral microbe identification microarray. Journal of periodontology 80, 1421-1432 (2009).
20. Walters, J. & Lai, P. C. Should Antibiotics Be Prescribed to Treat Chronic Periodontitis? Dental clinics of North America 59, 919-933 (2015).
21. Santos, R. S., et al. The use of systemic antibiotics in the treatment of refractory periodontitis: A systematic review. Journal of the American Dental Association 147, 577-585 (2016).
22. Jepsen, K. & Jepsen, S. Antibiotics antimicrobials: systemic and local administration in the therapy of mild to moderately advanced periodontitis. Periodontology 2000 71, 82-112 (2016).
23. Yilmaz, O., Verbeke, P., Lamont, R. J. & Ojcius, D. M. Intercellular spreading of Porphyromonas gingivalis infection in primary gingival epithelial cells. Infection and immunity 74, 703-710 (2006).
24. Blaser, M. J. & Falkow, S. What are the consequences of the disappearing human microbiota? Nature reviews. Microbiology 7, 887-894 (2009).
25. Fisher, S., et al. Progression of periodontal disease in a maintenance population of smokers and non-smokers: a 3-year longitudinal study. Journal of periodontology 79, 461-468 (2008).
26. Claffey, N., Polyzois, I. & Ziaka, P. An overview of nonsurgical and surgical therapy. Periodontology 2000 36, 35-44 (2004).
27. Uzel, N. G., et al. Microbial shifts during dental biofilm re-development in the absence of oral hygiene in periodontal health and disease. Journal of clinical periodontology 38, 612-620 (2011).
28. Ho, M. H., Lamont, R. J. & Xie, H. A novel peptidic inhibitor derived from Streptococcus cristatus ArcA attemiates virulence potential of Porphyromonas gingivalis. Scientific reports 7, 16217 (2017).
29. Mantri, C. K., et al. Fimbriae-mediated outer membrane vesicle production and invasion of Porphyromonas gingivalis. MicrobiologyOpen 4, 53-65 (2015).
30. Ho, M. H., et al. Two Small Molecules Block Oral Epithelial Cell Invasion by Porphyromons gingivalis. PloS one 11, e0149618 (2016).
31. Huang, L., et al. Picomolar dichotomous activity of gnidimacrin against HIV-1. PloS one 6, e26677 (2011).
32. Lai, W., et al. Gnidimacrin, a Potent Anti-HIV Diterpene, Can Eliminate Latent HIV-1 Ex Vivo by Activation of Protein Kinase C beta. Journal of medicinal chemistry 58, 8638-8646 (2015).

### H. CONCLUSIONS

It is to be understood that any given elements of the disclosed embodiments of the invention may be embodied in a single structure, a single step, a single substance, or the like. Similarly, a given element of the disclosed embodiment may be embodied in multiple structures, steps, substances, or the like.

The foregoing description illustrates and describes the processes, machines, manufactures, compositions of matter, and other teachings of the present disclosure. Additionally, the disclosure shows and describes only certain embodiments of the processes, machines, manufactures, compositions of matter, and other teachings disclosed, but, as mentioned above, it is to be understood that the teachings of the present disclosure are capable of use in various other combinations, modifications, and environments and is capable of changes or modifications within the scope of the teachings as expressed herein, commensurate with the skill and/or knowledge of a person having ordinary skill in the relevant art. The embodiments described hereinabove are further intended to explain certain best modes known of practicing the processes, machines, manufactures, compositions of matter, and other teachings of the present disclosure and to enable others skilled in the art to utilize the teachings of the present disclosure in such, or other, embodiments and with the various modifications required by the particular applications or uses. Accordingly, the processes, machines, manufactures, compositions of matter, and other teachings of the present disclosure are not intended to limit the exact embodiments and examples disclosed herein. Any section headings herein are provided only for consistency with the suggestions of 37 C.F.R. § 1.77 or otherwise to provide organizational queues. These headings shall not limit or characterize the invention(s) set forth herein.

What is claimed:

1. A pharmaceutical composition for inhibiting invasion of a pathogen into a host cell, comprising: a therapeutically effective amount of a protein kinase C beta (PKC-β) agonist and a pharmaceutically acceptable carrier, wherein the pharmaceutical composition is formulated for local delivery to a gingival tissue in a subject.

2. The pharmaceutical composition of claim 1, wherein the PKC-β agonist comprises gnidimacrin or a pharmaceutically acceptable derivative thereof.

3. The pharmaceutical composition of claim 1, wherein the therapeutically effective amount of the PKC-β agonist is sufficient to achieve a concentration of up to about 100 nM gnidimacrin in the gingival tissue of the subject.

4. The pharmaceutical composition of claim 1, wherein the pathogen comprises a bacterium and the bacterium is Porphyromonas gingivalis.

5. The pharmaceutical composition of claim 1, further comprising a therapeutically effective amount of an antibiotic.

6. The pharmaceutical composition of claim 1, wherein the pharmaceutical composition is formulated in a dentifrice.

7. The pharmaceutical composition of claim 6, wherein the dentifrice comprises a paste, a gel, a mouthwash, a powder, a tooth soap, or a combination thereof.

8. A pharmaceutical composition for treating or preventing periodontitis or a periodontitis-related condition or symptom in a subject, comprising:
a therapeutically effective amount of gnidimacrin or a pharmaceutical derivative thereof;
a therapeutically effective amount of an antibiotic; and
a pharmaceutically acceptable carrier,
wherein the pharmaceutical composition is formulated for local delivery to gingival tissue in the subject.

9. The pharmaceutical composition of claim 8, wherein the therapeutically effective amount of the gnidimacrin or the pharmaceutical derivative thereof is sufficient to achieve a concentration of about 10 nM to about 100 nM gnidimacrin or pharmaceutical derivative thereof in the gingival tissue of the subject.

10. The pharmaceutical composition of claim 8, wherein the antibiotic comprises gentamicin, metronidazole, or a combination thereof.

11. The pharmaceutical composition of claim 8, wherein the gnidimacrin or the pharmaceutical derivative thereof is formulated in a dentifrice and the dentifrice comprises a paste, a gel, a mouthwash, a powder, a tooth soap, or a combination thereof.

12. A method of treating or preventing periodontitis, a periodontitis-related condition, or a periodontitis-related symptom in a gingival tissue of a subject in need thereof, comprising:
locally administering the pharmaceutical composition of claim 1 to the gingival tissue of the subject.

13. The method of claim 12, wherein the periodontitis, periodontitis-related condition, or periodontitis-related symptom is mediated by Porphyromonas gingivalis.

14. The method of claim 12, wherein the compound is administered as a paste, a gel, a mouthwash, a powder, a tooth soap, or a combination thereof.

15. The method of claim 12, wherein the compound comprises gnidimacrin or a pharmaceutically acceptable derivative thereof.

16. A method of reducing the likelihood of invasion of a host cell by an intracellular bacterium, comprising:
contacting the host cell with an effective concentration of a PK-β agonist.

17. The method of claim 16, further comprising contacting the host cell with an effective concentration of an antibiotic.

18. The method of claim 16, wherein the PKC-β agonist comprises gnidimacrin or a pharmaceutical derivative thereof.

19. The method of claim 16, wherein the bacterium is Porphyromonas gingivalis.

20. The method of claim 16, wherein the host cell comprises an epithelial cell, an endothelial cell, a fibroblast, a smooth muscle cell, or a combination thereof.
